(19) United States
(12) Patent Application Publication
(10) Pub. No.: US 2025/0213652 A1
(43) Pub. Date: Jul. 3, 2025

(54) COMPOSITIONS AND THEIR USE IN METHODS FOR TREATING INTESTINAL INFLAMMATION

(71) Applicant: REVOLO BIOTHERAPEUTICS LIMITED, Great Shelford, Cambridgeshire (GB)

(72) Inventors: Roly Foulkes, Great Shelford, Cambridgeshire (GB); Jorge DeAlba, Great Shelford, Cambridgeshire (GB)

(21) Appl. No.: 18/852,312

(22) PCT Filed: Mar. 31, 2023
(86) PCT No.: PCT/GB2023/050876

§ 371 Date: Sep. 27, 2024

Related U.S. Application Data
(60) Provisional application No. 63/326,108, filed on Mar. 31, 2022.

(51) Int. Cl. A61K 38/17; A61K 9/00; A61P 1/04

(52) U.S. Cl. CPC A61K 38/1774; A61K 9/0019; A61P 1/04

(57) ABSTRACT

Described herein are methods for preventing or treating an inflammatory bowel disease (IBD) by administrating a binding immunoglobulin protein (BiP) or a functional analog thereof.

### FIELD OF THE INVENTION

The embodiments of the present invention relate to compositions and methods for the treatment of inflammatory bowel disease (IBD) with the binding immunoglobulin protein (BiP) or functional analogs thereof.

### BACKGROUND OF THE INVENTION

Inflammatory bowel disease (IBD), represented mainly by ulcerative colitis and Crohn disease but also including noninfectious inflammations of the bowel, have posed an enigma to gastroenterologists and immunologists alike since their first modern descriptions some 75-100 years ago. IBD is characterized by chronic inflammation of the gastrointestinal (GI) tract, resulting in damage to the GI tract. In 2017, there were 6.8 million cases of IBD globally. The burden of IBD is rising globally; the age-standardized prevalence rate increased from 79.5 per 100,000 population in 1990 to 84.3 per 100,000 population in 2017.¹ Most people with IBD are diagnosed in their 20s and 30s. Adults with IBD have higher health care use than those without IBD, including doctors' visits, medication prescriptions, emergency department visits, hospitalizations, and surgeries.²

IBD treatment usually involves either drug therapy or surgery. Anti-inflammatory drugs are often the first step in the treatment of inflammatory bowel disease (e.g., corticosteroids and aminosalicylates, such as mesalamine, balsalazide, and olsalazine). However, anti-inflammatory drugs are often insufficient to provide adequate and sustainable relief of IBD. Alternative therapies include immunosuppressant drugs (e.g., azathioprine, mercaptopurine, and methotrexate), biologics directed toward neutralizing proteins that cause inflammation (e.g., infliximab (Remicade), adalimumab (Humira), golimumab (Simponi), certolizumab (Cimzia), vedolizumab (Entyvio) and ustekinumab (Stelara)), and, as a last resort, surgery. Over the past two decades, advances in biologic and small molecule therapeutics have resulted in a rapid increase in the armamentarium of therapies for IBD. Despite these advancements, Crohn's disease and ulcerative colitis remain chronic and progressive diseases. One of the primary reasons for persistent inflammation and bowel damage is failure of current medical therapy.

Currently, there is no cure or effective treatment for patients diagnosed with functional gastrointestinal diseases such as IBD. The main goal of current therapies for IBD is to induce a clinical remission by focusing on symptoms and then maintain it for a long period of time, in order to realize the best attainable quality of life. As current therapies have limited efficacy, new therapies for treating inflammatory and chronic pain in the gut of subjects with IBD are required. Accordingly, there is a need for improved therapies for the treatment of IBD.

### BRIEF SUMMARY OF THE INVENTION

The embodiments of the present invention provide a method for modulating an inflammatory response associated with intestinal inflammation. In some embodiments, the embodiments of the present invention provide a method of preventing or treating an inflammatory bowel disease (IBD) comprising administering to a subject at risk of, or afflicted with, the IBD a therapeutically effective amount of a binding immunoglobulin protein (BiP) or a functional analog thereof. In some embodiments, the BiP or functional analog thereof has an amino acid sequence selected from the group consisting of: SEQ ID NO:1, SEQ ID NO:2, SEQ ID NO: 3, SEQ ID NO:4, and SEQ ID NO:5, or a conservative amino acid substituent thereof. In alternate embodiments, the BiP or functional analog thereof has an amino acid sequence consisting of: SEQ ID NO:1. In some embodiments, the IBD is ulcerative colitis, Crohn's disease, or a noninfectious inflammation of the bowel.

In some embodiments, the therapeutically effective amount of a BiP-related polypeptide is administered once monthly. In some embodiments, the BiP-related polypeptide is administered every 1, 2, 3, 4, 6, or 8 weeks. In some embodiments, the BiP-related polypeptide is administered intermittently, with administration starting at the onset of IBD symptoms and the administration stopping upon remission of the IBD symptoms.

In some embodiments, the therapeutically effective amount of a BiP-related polypeptide is 1-30 mg, 5-25 mg, 10-20 mg, 12-18 mg, or 15 mg, IV. In alternative embodiments, the therapeutically effective amount of a BiP-related polypeptide is 25-75 mg, 30-70 mg, 40-60 mg, 45-55 mg, or 50 mg, IV. In yet other alternative embodiments, the therapeutically effective amount of a BiP-related polypeptide is 50-200 mg, 75-150 mg, 85-125 mg, 90-110 mg, 95-105 mg, or 100 mg IV.

Other implementations are also described and recited herein.

### BRIEF DESCRIPTION OF THE DRAWINGS

For the purpose of illustration, certain embodiments of the present invention are shown in the drawings described below. It should be understood, however, that the invention is not limited to the precise arrangements, dimensions, and instruments shown. In the drawings:

FIG. 1 provides the amino acid sequence of IRL201805 (SEQ ID NO:1), a BiP analog.³

FIG. 2 provides the amino acid sequence of a His-tagged BiP analog expressed by plasmid pQE2 (SEQ ID NO:2).⁴

FIG. 3A-D provides the amino acid sequence of a BiP analog (SEQ ID NO:3).⁵

FIG. 4A-D provides the amino acid sequence of a BiP analog (SEQ ID NO:4).⁶

FIG. 5 provides the amino acid sequence of native BiP (SEQ ID NO:5).⁷

FIG. 6A-E provides an outline of the procedure used to assess the effects of human primary immune cells ex vivo within inflamed and non-inflamed colon tissue treated with IRL201805.

FIG. 7 shows the effect of 12 hour treatment with 20 μg/mL IRL201805 on CD69+/CD39+ Tregs in inflamed and non-inflamed colon tissues from IBD patients (Crohn's disease or Ulcerative colitis), as determined by flow cytometry.

FIG. 8 depicts the mean fluorescent intensity of surface expression of CD69 and CD39 on Tregs from peripheral blood mononuclear cells (PBMCs) cultured with 20 μg/mL IRL201805 for 20-72 h at 37° C.

FIG. 9A-B illustrates the effects of 20 g/mL IRL201805 on JAK-STAT signaling pathway at the transcript and protein level. FIG. 9A provides the RNAseq analysis of gene transcript levels of key signaling genes involved in CD69 activation. FIG. 9B provides the immunoblot of p-STAT5 levels in cell lysates from untreated and IRL1805 treated PBMCs.

FIG. 10A provides a schematic representation of CD69 relevant ligands.

FIG. 10B provides a bar graph showing the effects of IRL201805 on CD69 ligands, galectin-1 (Gal-1) and S100A8/A9, as detected by TMT quantitative proteomics. FIG. 10B provides a diagrammatic representation of the increased trimethylation post-translational modifications on threonine 113 on S100A9 post-IRL201805 treatment.

FIG. 11A-C provides bar graphs showing the effects of IRL201805 on the expression of surface CD154 and CD69 activation markers on T-lymphocytes from PBMCs: Tregs (FIG. 11A); effector T cells (Teffs; FIG. 11B); and CD8 T cells (FIG. 11C). Results are from PBMCs isolated from four healthy donors. Mean±SD shown. P=values represent paired sample t-test. NS=not significant.

FIG. 12 provides a schematic representation of the effects of IRL201805 on CD69 and CD69 relevant ligands. Abbreviations: APC, antigen presenting cell; TCR, T cell receptor; Treg, regulatory T-cell; P, phosphorylation.

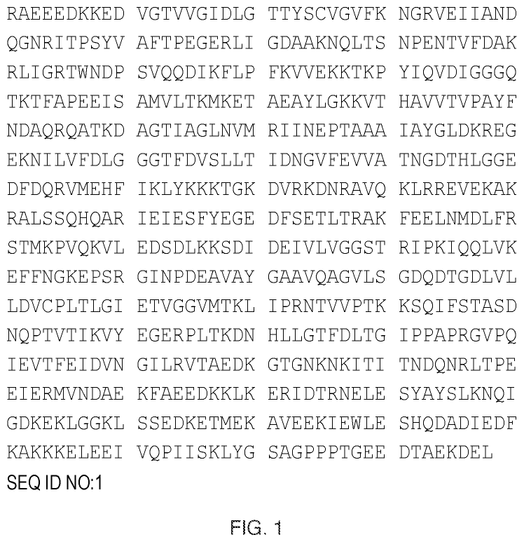

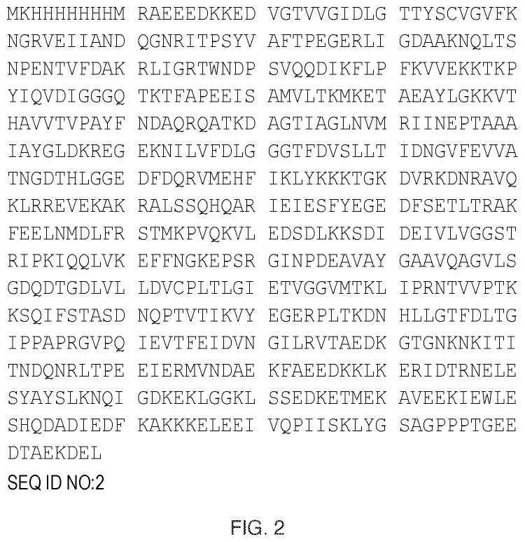

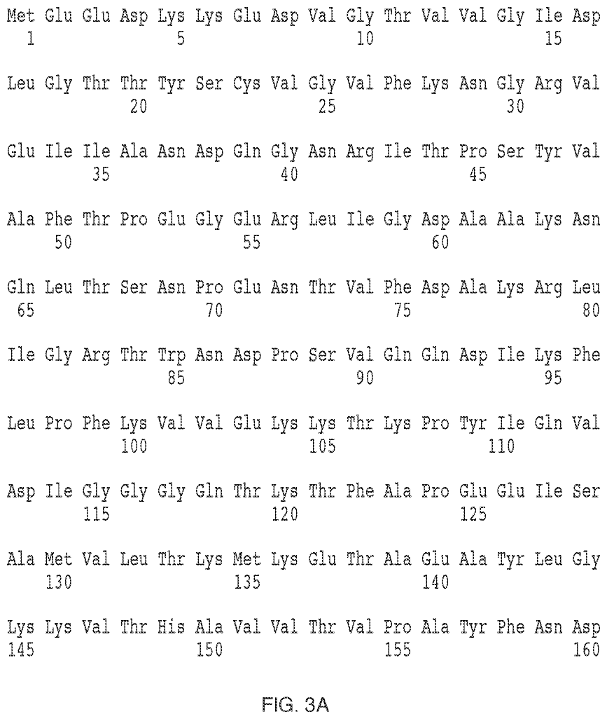

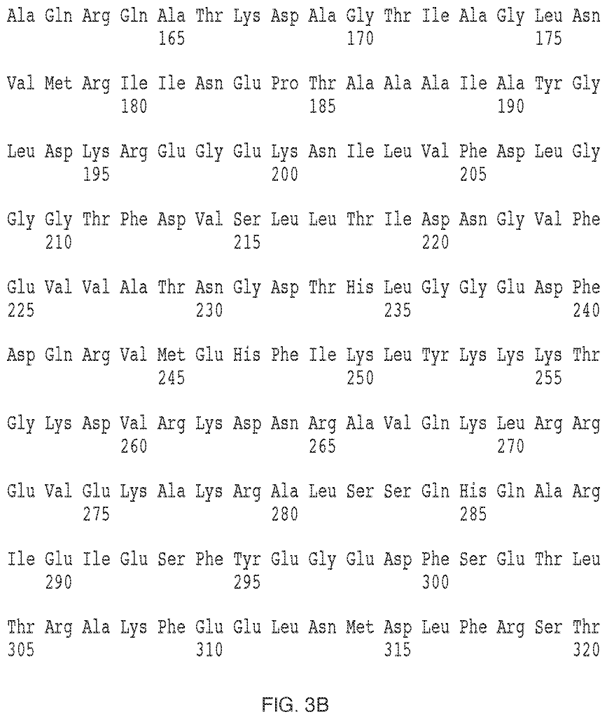

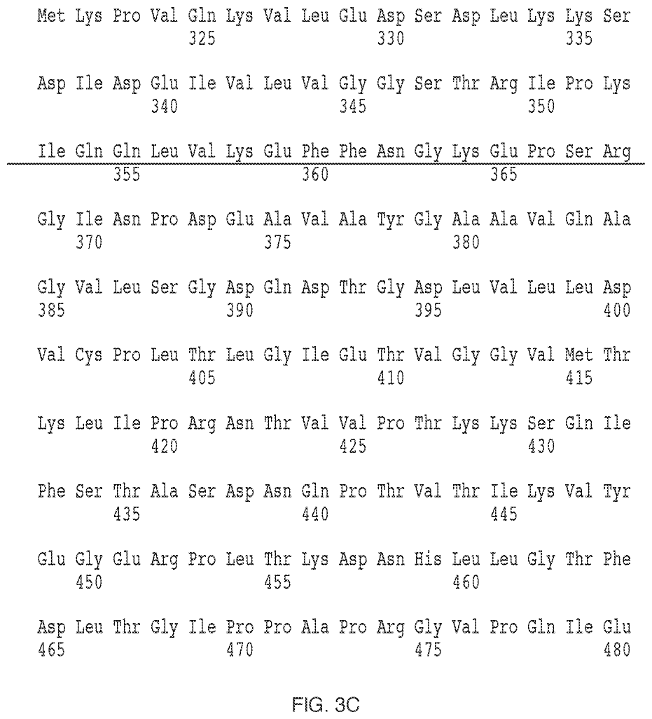

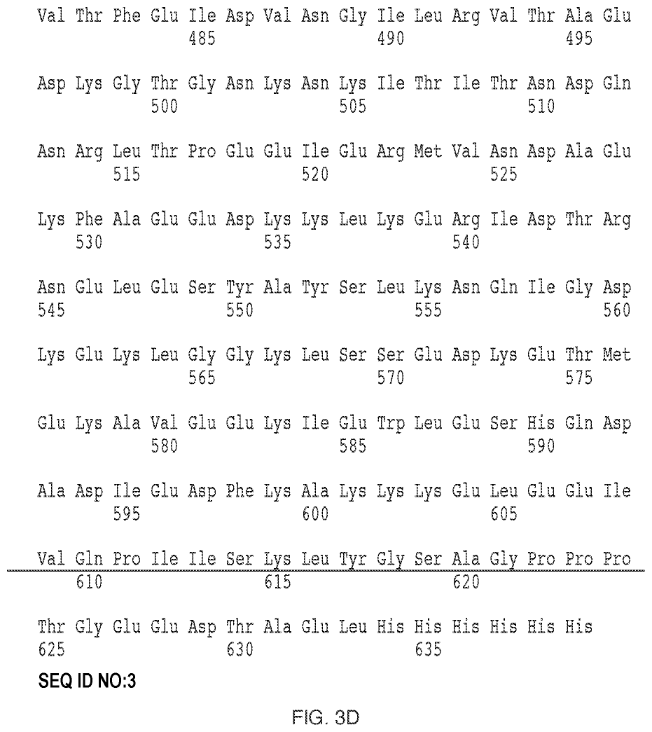

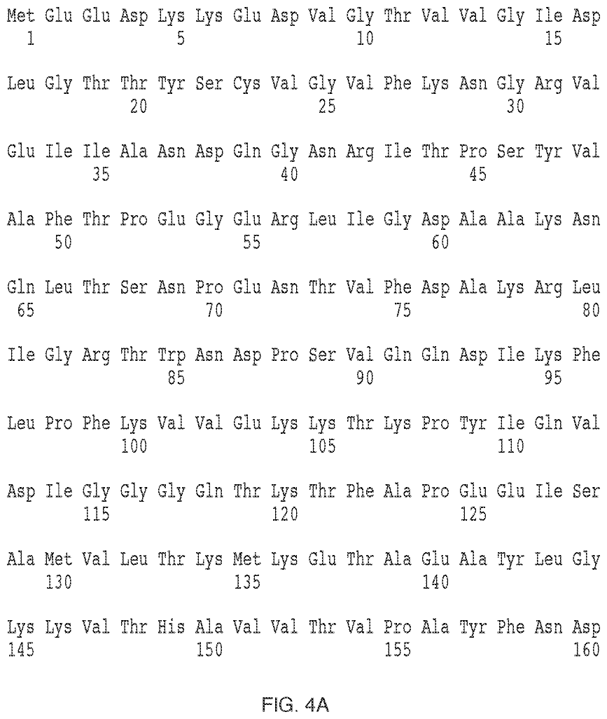

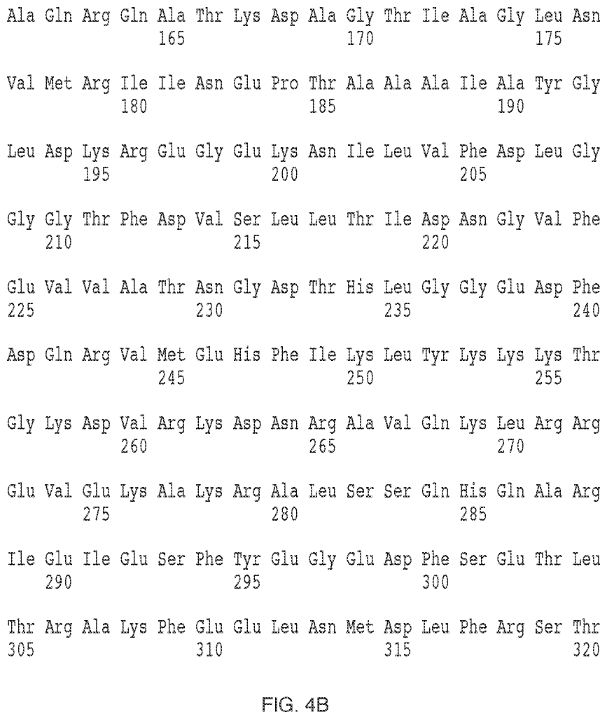

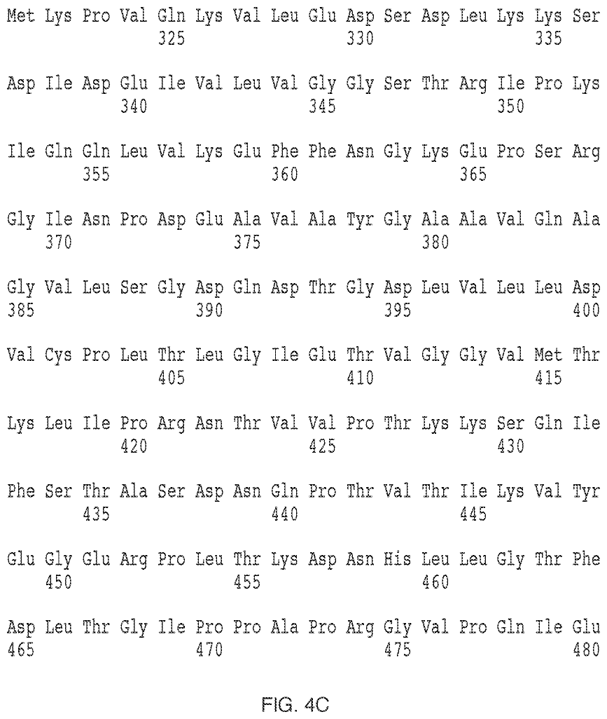

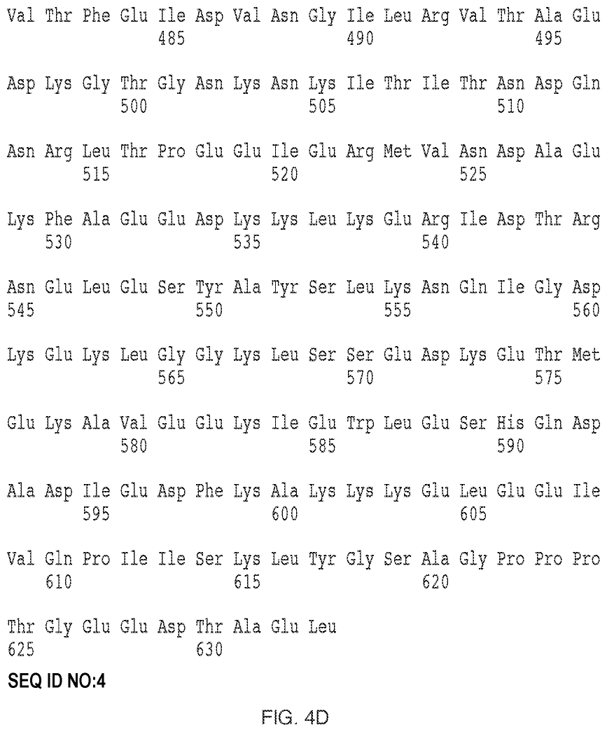

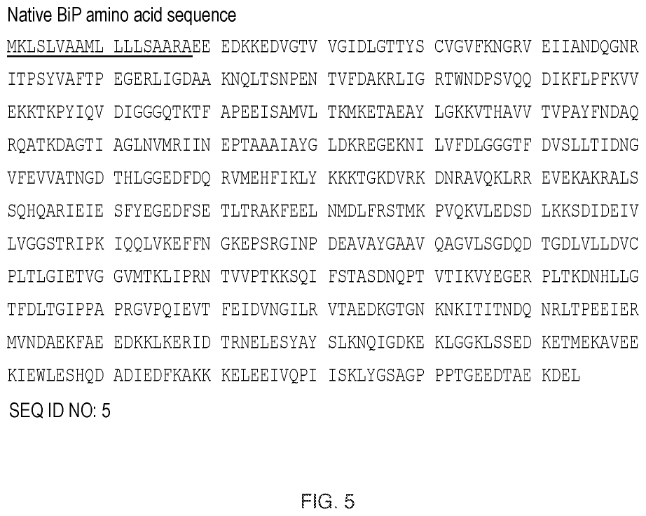

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex.

As used herein, the terms “effective amount” and “therapeutically-effective amount” include an amount sufficient to prevent or ameliorate a manifestation of disease or medical condition, such as an immune disorder including rheumatoid arthritis, psoriatic arthritis, juvenile idiopathic arthritis, ankylosing spondylitis, rejection of a transplant of an organ, skin, tissue, blood, serum, plasma, inflammatory bowel disease, or Crohn's disease. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treating” or “treatment” or “to treat” or “alleviating” or “to alleviate” refer to both (1) therapeutic measures that cure, slow down, lessen symptoms of, and/or halt progression of a diagnosed disease or infection and (2) prophylactic or preventative measures that prevent or slow the development of a disease or infection. Symptoms of IBD may include, but are not limited to, persistent diarrhea, abdominal pain, rectal bleeding/bloody stools, urgency to have a bowel movement and fecal incontinence, fatigue, fever, anemia, and/or weight loss. Malnutrition and delayed growth can also occur in subjects who develop IBD as children.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of an immune disorder, delay or slowing of an immune disorder, and an increased lifespan as compared to that expected in the absence of treatment.

As used herein, the term “intermittent” administration means that the therapeutic agent or drug is administered upon the presence of IBD symptoms and the administration is stopped upon remission of the IBD symptoms.

As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

The terms “decrease,” “reduced,” “reduction,” or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, a nucleic acid encoding a polypeptide as described herein (e.g., an antibody or antibody reagent) is comprised by a vector. In some of the aspects described herein, a nucleic acid sequence encoding a given polypeptide as described herein, or any module thereof, is operably linked to a vector. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion thereof, or chimeric antigen receptor (CAR) described herein is isolated. In some embodiments, the antibody, antibody reagent, antigen-binding portion thereof, or CAR described herein is purified.

As used herein, “engineered” refers to the aspect of having been manipulated by the hand of man. For example, an antibody, antibody reagent, antigen-binding portion thereof, CAR or bispecific antibody is considered to be “engineered” when the sequence of the antibody, antibody reagent, antigen-binding portion thereof, CAR or bispecific antibody is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragées, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragées, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically-acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher et al. (1996).⁸

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, l-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, l-pyroglutamic acid, salicylic acid, sebacic acid, stearic acid, succinic acid, sulfuric acid, l-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;⁹ The Encyclopedia of Molecular Cell Biology and Molecular Medicine;¹⁰ Molecular Biology and Biotechnology: a Comprehensive Desk Reference;¹¹ Immunology;¹² Janeway's Immunobiology;¹³ Lewin's Genes XI;¹⁴ Molecular Cloning: A Laboratory Manual;¹⁵ Basic Methods in Molecular Biology;¹⁶ Laboratory Methods in Enzymology;¹⁷ Current Protocols in Molecular Biology (CPMB);¹⁸ Current Protocols in Protein Science (CPPS);¹⁹ and Current Protocols in Immunology (CPI).²⁰

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### Inflammatory Bowel Disease (IBD)

The enteric nervous system (ENS) controls or regulates vital gastrointestinal functions, including motility, secretion, local immunity, and inflammation, and represents the largest collection of autonomous neurons outside of the brain. Disorders involving the ENS (e.g., IBD) are common and major contributors to the health burden throughout the world.

IBD, associated with damage to the ENS, is characterized by chronic severe inflammation of the small bowel and/or the colon leading to recurrent diarrhea and abdominal pain. Crohn disease (CD) and ulcerative colitis (UC) are the two main clinicopathological subtypes of IBD. Despite both being chronic and relapsing inflammatory diseases of the bowel, they can be differentiated by the location of the inflammation in the gastrointestinal tract and by the nature of the histological alterations in the intestinal wall. Anatomically, CD can affect the entire gastrointestinal tract from mouth to anus, although it commonly affects the terminal ileum and colon. UC is restricted to the rectum, colon and caecum. Microscopically, CD is transmural and often discontinuous while UC affects only the intestinal mucosa in a continuous pattern.

IBD is a very disabling disease due to the fatigue associated with the inflammatory symptoms and due to the chronic pain suffered by patients. The pathogenesis of IBD is only partially understood; various environmental and host (e.g., genetic, epithelial, immune and nonimmune) factors are involved. Complex interactions between the immune system, enteric commensal bacteria/pathogens and host genotype are thought to underlie the development of IBD. These relapsing chronic inflammatory disorders appear to be caused by overly aggressive T-cell responses directed against environmental factors and/or a subset of commensal bacteria/pathogens that inhabit the distal ileum and colon of genetically susceptible hosts.

Currently, there is no cure or effective treatment for patients diagnosed with functional gastrointestinal diseases such as IBD. The main goal of current therapies for IBD is to induce a clinical remission by focusing on symptoms and then maintain it for a long period of time, in order to realize the best attainable quality of life. As current therapies have limited efficacy, new therapies for treating inflammatory and chronic pain in the gut of subjects with IBD is clinically significant.

### Binding Immunoglobulin Protein (BiP) and Related Polypeptides

It is known that intracellular BiP is an anti-apoptotic protein with binding preference for hydrophobic residues exposed by both denatured or unfolded nascent polypeptides. BiP correctly folds nascent polypeptides but also protects cells by binding denatured proteins and, as an endoplasmic reticulum (ER) resident protein, is tethered in the ER by the KDEL amino acid sequence at the 3′ terminus of the protein which binds to ERD2.

Natural endogenous BiP, also known as glucose-regulated protein 78 (GRP78), is a stress protein (SP) and member of the heat shock protein (HSP) 70 family. Present in all nucleated cells, BiP is released from sequestration and upregulated on activation of the unfolded protein response (UPR). This is initiated by the intracellular accumulation of denatured protein and nascent polypeptides caused particularly by cell stresses, such as reduced oxygen and/or glucose and an increase in reactive oxygen species. These conditions are features of the pathology of chronic inflammation such as in the inflamed synovium of patients with rheumatoid arthritis (RA).

In common with other SP, or HSP, with vital intracellular functions, these molecular chaperones, including BiP, are expressed on the cell surface and secreted on upregulation, allowing unexpected and distinctly different extracellular functions. This has raised awareness that these highly conserved proteins may provide a link between the innate and adaptive immune systems.

IRL201805 (or 1805) is a modified analog of GRP78 being developed for the treatment of autoimmune disease. During extensive investigations, it has been shown that rhuBiP (IRL201805 lacking the C-terminal KDEL sequence), can treat and inhibit disease progression in animal models of arthritis. Adoptive cell transfer studies using spleen and lymph node cells from rhuBiP/IRL201805-treated mice, in the collagen induced arthritis (CIA) model, show that rhuBiP/IRL201805 has therapeutic properties that are long-lived in the absence of rhuBiP/IRL201805 and are related to changes in the immune cell population.

Thus, rhuBiP/IRL201805 changes the cellular immune response towards an anti-inflammatory TH2 profile and immune homeostasis. Further work following biomarkers in patients have confirmed increased stabilization and efficacy of regulatory T cells and a tendency to divert dendritic cell differentiation to an Indoleamine 3, 2 oxygenase (IDO)+, more anti-inflammatory, phenotype.

These data indicate that IRL201805 has profound activity in reducing the inflammatory response associated with rheumatoid arthritis, however, its mode of action suggests potential efficacy in a range of immune based disorders.

CD69 is a transmembrane glycoprotein with a C-type lectin domain (CTLD)^{21,22,23} that has recently been identified as a crucial regulator of intestinal inflammation and a new target molecule for IBD treatment.²⁴ CD69 is not expressed in detectable levels on naive leukocytes, but its surface expression is induced promptly upon activation.^{25,26} In human diseases, CD69 expression is increased on leukocytes at the site of inflammation.^{27,28,29} Furthermore, early in vitro studies described CD69 as a proinflammatory molecule whose engagement induced intracellular Ca²⁺ influx, lymphocyte proliferation, and the production of proinflammatory mediators, such as IL-2, tumor necrosis factor- (TNF-) α, and nitric oxide (NO).^{30,31,32,33} CD69 is also necessary for the cell-contact dependent stimulation of macrophages by T cells.³⁴ However, recent in vivo studies with transgenic mice showed that CD69 can limit the immune response and proposed a regulatory function of CD69. CD69 has been shown to have a role in leukocyte migration, in the function of regulatory T cells and resident tissue memory T cells.³⁵ It has thus been suggested that the stable induction of CD69 expression should lead to the reduced lymphocyte migration to intestinal LP and to the generation of CD69⁺ Treg cells.³⁶

As described below, the present disclosure provides evidence that BiP-related polypeptides induce CD69 expression and CD69 relevant ligands, thus providing a method for modulating an inflammatory response associated with intestinal inflammation and a method of preventing or treating an inflammatory bowel disease (IBD).

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Example 1 Evaluation of the Efficacy of Binding Immunoglobulin Protein (BiP) Analogs in a Murine Model of Intestinal Inflammation

There currently exist more than 50 animal models of human IBD. While no one model perfectly phenocopies human IBD, many are useful for studying various aspects of human disease, including disease onset and progression and the wound-healing response.

In the present study, intestinal inflammation is initiated with syngeneic splenic CD4⁺CD45RB^{high} T cell adoptive transfer into T and B cell deficient recipient mice.³⁷ The CD4⁺CD45RB^{high} T cell population contains mainly naïve T cells primed for activation that are capable of inducing chronic small bowel and colonic inflammation. This animal model allows the modification of key experimental variables, including both innate and adaptive immune cell populations, to answer biologically relevant questions relating to disease pathogenesis. Additionally, it provides precise initiation of disease onset and a well-characterized experimental time course, which permits the kinetic study of clinical features of disease progression in mice. Intestinal inflammation induced by this method shares many features with human IBD, including chronic large and small bowel transmural inflammation, pathogenesis driven by cytokines such as TNF and IL-12, and systemic symptoms such as wasting.³⁸ Thus, it is an ideal model system to evaluate the efficacy of BiP analogs such as IRL201805 (SEQ ID NO:1).

### Materials and Methods

### Materials

IRL201805 (SEQ ID NO:1) is made to GMP standard by CBC, Bristol, UK.

### Methods

### Experimental Outline

Male RAG2^{-/-} mice are randomly allocated to experimental groups and allowed to acclimatize for a week. On Day 0, spleens are removed from donor C57Bl/6N male mice and single cell suspensions generated according to CRL SOPs. On Day 0, male RAG KO mice are injected intraperitoneally with CD4⁺CD45RB^{high} T cells (CD45RB^{low} T cells for group 1 only) resuspended in 500 μL PBS.

From Day 21 until the end of the experiment on Day 49, animals are monitored daily for clinical signs of colitis to include: bodyweight loss, loose stools and/or diarrhea. On Day 49, animals are culled, the colon dissected out, and colon weight and length measured. Colons from half the animals in each group are transferred in tissue fixative, then processed for paraffin embedding and stored for histopathology analysis. Lamina propria from half the animals in each group are processed for flow cytometry.

### Administration Schedule

Treatments are administered in accordance with the schedule in Table 1 below. All Groups are n=10.

TABLE 1

| | Treatments | | | |
|---|---|---|---|---|
| Groups | Dose | Route | Regimen | Intervention |
| Untreated | NA | NA | NA | CD45RB^{low} |
| (n = 6) | | | | T cells |
| | | | | suspended in |
| | | | | 500 μL PBS IP |
| Vehicle | NA | IV | Day 21 to end, 1 × weekly | CD45RB^{high} |
| Abatacept | CRL | IP | Day 21 to end, 3 × weekly | T cells |
| IRL201805 | TBC | IV | Day 21 to end, 1 × weekly | suspended in |
| IRL201805 | TBC | IV | Day 21, once | 500 μL PBS IP |

Abbreviations:
NA: not applicable,
IP: intra-peritoneal injection,
IV: intra-venous injection,
TBC: to be confirmed,
CRL: Charles River regime.

### Morbidity and Mortality

Morbidity and mortality are assessed daily. Animals are monitored for clinical signs to include abnormal posture (e.g., hunched), abnormal coat condition (e.g., piloerection), changes to the colour of hairless areas (including cyanosis or jaundice), presence of masses and/or swelling, abnormal breathing, abnormal movements and decrease activity. Cause of death are noted whenever identified. Survival rates are recorded.

### Bodyweights

Animals are weighed at the start of the study (Day 0), and regularly for the first three weeks. From Day 21 until the end of the study, all animals are weighed daily. Data are analyzed and graphed.

### Clinical Scores

From Day 21 until the end of the experiment, animals are monitored daily for clinical signs of colitis to include bodyweight loss, loose stools and/or diarrhea. The following scoring system are provided in Table 2.

TABLE 2

| Score | Weight Loss | Stool Consistency |
|---|---|---|
| 0 | None | Normal |
| 1 | 1-5% | |
| 2 | 5-10% | Loose |
| 3 | 10-20% | |
| 4 | >20% | Diarrhea |

### Flow Cytometry

Single cell suspensions are prepared from colon lamina propria cells isolated from half the animals in each group using the manufacturer's protocol (Miltenyi Biotec). Cells are harvested, restimulated with PMA/lonomycin, and stained with the following panel containing a viability dye and surface and intracellular markers: CD44, CD45, CD3, CD4, IFNγ, IL-17, CD25 and FOXP3. Counts and percentages for each population are determined.

### Gross Pathology

At termination, colon are dissected out and a picture is taken to allow for length measurements to be performed on digital images using the Image J software. Colons are also weighed.

### Histopathology

At termination, colon from half the animals in each group are transferred in tissue fixative, then processed for paraffin-embedding in a ‘swiss roll’ format. These samples are sectioned and stained with hematoxylin and eosin (H&E) and up to two sections per animal are scored by a qualified histopathologist according to a semi-quantitative scoring system. Sections are scored for mucosal thickness, mucosal ulceration, lamina propria mononuclear cell infiltration, lamina propria granulocyte infiltration and crypt abscesses/dilation/distortion as described in Table 3. An overall severity score are calculated by adding the scores for each of the criterion.

TABLE 3

| Parameter | Criterion | Scores |
|---|---|---|
| Mucosal thickness | Normal | 0 |
| | Mildly increased | 1 |
| | Markedly increased | 2 |
| Mucosal ulceration | None | 0 |
| | Erosions | 1 |
| | Ulcers | 2 |
| Lamina propria mononuclear | Normal | 0 |
| cell infiltration | Mild | 1 |
| | Severe | 2 |
| Lamina propria granulocyte | Normal | 0 |
| infiltration | Mild | 1 |
| | Severe | 2 |
| Crypt abscesses/dilation/distortion | None | 0 |
| | Few | 1 |
| | Many | 2 |

### Results

In the present study, IRL201805 results in a significant reduction in clinical signs of colitis including a reduction in bodyweight loss, loose stools and/or diarrhea, as well as in a significant reduction in severity scores bases on sections scored for mucosal thickness, mucosal ulceration, lamina propria mononuclear cell infiltration, lamina propria granulocyte infiltration and crypt abscesses/dilation/distortion.

These results suggest that administering a binding immunoglobulin protein (BiP), or a functional analog, to a subject at risk of, or afflicted with, the IBD is an effective method of preventing or treating IBD. BiP or functional analogs useful in the methods of the present intention include, but are not limited to, polypeptides having an amino acid sequence selected from SEQ ID NOS: 1-5.

### Example 2 Evaluation of the Efficacy of Binding Immunoglobulin Protein (BiP) Analogs in a Dextran Sulphate Sodium (DSS)-Induced Murine Model of Ulcerative Colitis (UC)

Perhaps the most widely used mouse model of colitis employs dextran sodium sulfate (DSS), a chemical colitogen with anticoagulant properties, to induce disease. DSS is a water-soluble, negatively charged sulfated polysaccharide with a highly variable molecular weight ranging from 5 to 1400 kDa. The most severe murine colitis, which most closely resembles human UC,³⁹ results from administration of 40-50 kDa DSS in drinking water. The mechanism by which DSS induces intestinal inflammation is unclear but is likely the result of damage to the epithelial monolayer lining the large intestine allowing the dissemination of proinflammatory intestinal contents (e.g., bacteria and their products) into underlying tissue. The DSS colitis model is very popular in IBD research due to its rapidity, simplicity, reproducibility and controllability. Acute, chronic and relapsing models of intestinal inflammation can be achieved by modifying the concentration of DSS and the frequency of administration.

In the present study, the DDS murine model of UC will be used to evaluate the efficacy of BiP analogs such as IRL201805 (SEQ ID NO:1).

### Materials and Methods

### Materials

IRL201805 (SEQ ID NO:1) is made to GMP standard by CBC, Bristol, UK.

### Methods

### Experimental Outline

Adult C57-bl/6 mice are randomly allocated to experimental groups and allowed to acclimatize for one week. From Day-2, treatments are administered in accordance with the administration schedule below. On Day 0, drinking water is replaced by a 5% dextran sulphate sodium (DSS) in water (excluding group 1 which remains on normal drinking water throughout). Animals are given ad libitum access to the 5% DSS solution. From Day 0 until the end of the experiment on Day 7, animals are monitored daily for clinical signs of colitis to include: bodyweight loss, loose stools and/or diarrhea and presence of occult or gross blood in the stools.

Blood samples are taken on Day −2, Day 3 (in-life) and Day 7 (terminal), processed to serum, and stored for analysis.

On Day 7, animals are culled, the colon dissected out, and colon length measured. One sample (distal) of colon per animal are taken and fixed for histopathology analysis. A further sample (proximal) of colon is taken at termination and cultured for 48 hours in the presence or absence of LPS prior to analysis of cytokines. Remaining supernatants are stored for analysis.

### Administration Schedule

Treatments are administered in accordance with the schedule in Table 1 below. All Groups are n=10 unless indicated otherwise.

TABLE 1

| | Treatments | | | |
|---|---|---|---|---|
| Groups | Dose | Route | Regimen | Intervention |
| Non-diseased, vehicle | NA | IV | Day −2, Day 0, Day 3, Day 6 | Normal drinking water |
| (n = 5) | | | (×4 total) | |
| Vehicle | NA | IV | Day −2, Day 0, Day 3, Day 6 | DSS 5%, Day 0 to End |
| | | | (×4 total) | |
| Reference compound - | 75 mg/kg | PO | SID day 0-6 | |
| Cyclosporin | | | | |
| Test compound 1805 | 1 mg/kg | IV | Day −2, Day 0, Day 3, Day 6 | |
| | | | (×4 total) | |

Abbreviations:
TBC: not applicable,
NA: not applicable,
IV: intravenous,
PO: per oral.

### Morbidity and Mortality

Morbidity and mortality are assessed daily. Animals are monitored for clinical signs to include abnormal posture (e.g., hunched), abnormal coat condition (e.g., piloerection), changes to the colour of hairless areas (including cyanosis or jaundice), presence of masses and/or swelling, abnormal breathing, abnormal movements and decrease activity. Cause of death are noted whenever identified. Survival rates are recorded.

### Bodyweights

From Day 1 until the end of the study, all animals are weighed daily. Data are analyzed and graphed.

### Clinical Scores

From Day 0 until the end of the experiment, animals are monitored daily for clinical signs of colitis to include bodyweight loss, loose stools and/or diarrhea and presence of blood, occult or gross, in the stools. The following scoring system are provided in Table 2.

TABLE 2

| Score | Weight Loss | Stool Consistency | Bleeding |
|---|---|---|---|
| 0 | None | Normal | None |
| 1 | 1-5% | | |
| 2 | 5-10% | Loose | Hemoccult |
| 3 | 10-20% | | |
| 4 | >20% | Diarrhea | Gross bleeding |

### Gross Pathology

At termination, colon are dissected out and a picture is taken to allow for length measurements to be performed on digital images using the Image J software. Colons are also weighed.

### Measurements of Colon Explant Cytokine Levels

Colon samples taken on Day 7 are cultured for 48 hours in the presence or absence of LPS prior to analysis of cytokine content by Luminex. The following cytokines are analyzed by multiplex (8-plex) Luminex: sTNFRII, SIL-6R, IL-6, IL-10, IL-1b, TNF-a, IFN-γ and MCP-1. Samples are analyzed in singlicate using multiplex xMAP bead technology, which utilizes microspheres as a solid support for sandwich immunoassays and determination of numerous cytokines in the same sample (Luminex).

### Histopathology

At termination, colon from half the animals in each group are transferred in tissue fixative, then processed for paraffin-embedding in a ‘swiss roll’ format. These samples are sectioned and stained with hematoxylin and eosin (H&E) and up to two sections per animal are scored by a qualified histopathologist according to a semi-quantitative scoring system. Sections are scored for mucosal thickness, mucosal ulceration, lamina propria mononuclear cell infiltration, lamina propria granulocyte infiltration and crypt abscesses/dilation/distortion as described in Table 3. An overall severity score are calculated by adding the scores for each of the criterion.

TABLE 3

| Parameter | Criterion | Scores |
|---|---|---|
| Mucosal thickness | Normal | 0 |
| | Mildly increased | 1 |
| | Markedly increased | 2 |
| Mucosal ulceration | None | 0 |
| | Erosions | 1 |
| | Ulcers | 2 |
| Lamina propria mononuclear | Normal | 0 |
| cell infiltration | Mild | 1 |
| | Severe | 2 |
| Lamina propria granulocyte | Normal | 0 |
| infiltration | Mild | 1 |
| | Severe | 2 |
| Crypt abscesses/dilation/distortion | None | 0 |
| | Few | 1 |
| | Many | 2 |

### Results

In the present study, IRL201805 results in a significant reduction in clinical signs of colitis including a reduction in bodyweight loss, loose stools and/or diarrhea, as well as in a significant reduction in severity scores bases on sections scored for mucosal thickness, mucosal ulceration, lamina propria mononuclear cell infiltration, lamina propria granulocyte infiltration and crypt abscesses/dilation/distortion.

These results suggest that administering a binding immunoglobulin protein (BiP), or a functional analog, to a subject at risk of, or afflicted with, the IBD is an effective method of preventing or treating IBD. BiP or functional analogs useful in the methods of the present intention include, but are not limited to, polypeptides having an amino acid sequence selected from SEQ ID NOS: 1-5,

### Example 3 Evaluation of the Effects of Binding Immunoglobulin Protein (BiP) Analogs on CD69 in Colon Punch Biopsies Obtained from IBD Patients

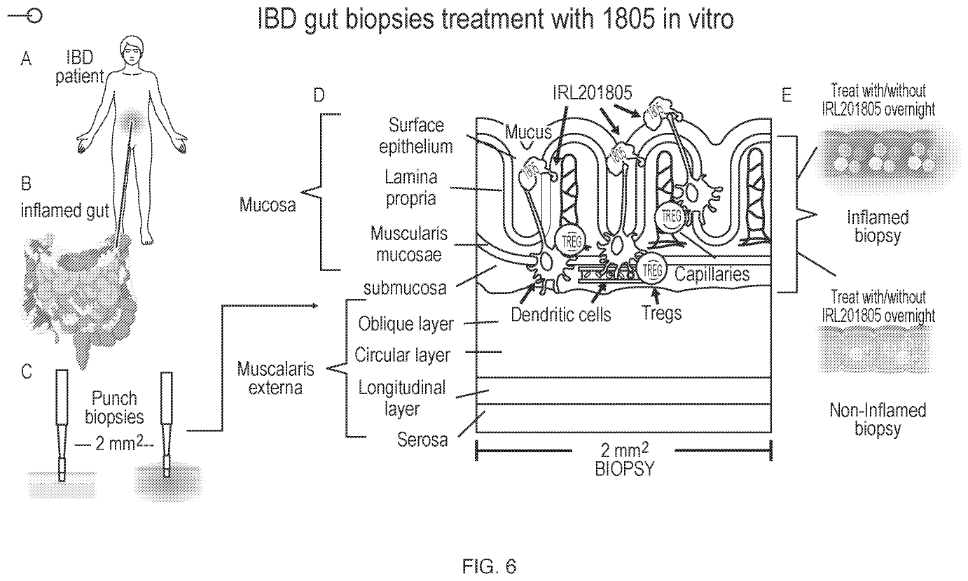

In the present study, punch biopsies (2 mm²) of inflamed and non-inflamed colon tissues were obtained from each consenting IBD patient with hospital ethical approval, as illustrated in FIG. 6A-E. Tissue biopsies were subsequently incubated for 12-14 hours with or without IRL201805 (20 μg/mL) and assessed for CD69+/CD39+ regulatory T cells (Tregs).

As described above, CD69 is known to have a number of biological functions. In regulatory T cells (Tregs), CD69 is thought to promote CD39 surface expression, FoxP3 transcription, and STAT5 phosphorylation leading to STAT3 inhibition. CD39 is an integral membrane protein that phosphohydrolyzes ATP, and less efficiently ADP, in a Ca²⁺- and Mg²⁺-dependent fashion, to yield AMP. Ultimately the adenosine which is generated is a potent activator of tolerogenic dendritic cells (DCs). CD39 becomes catalytically active upon its localization on the cell surface.

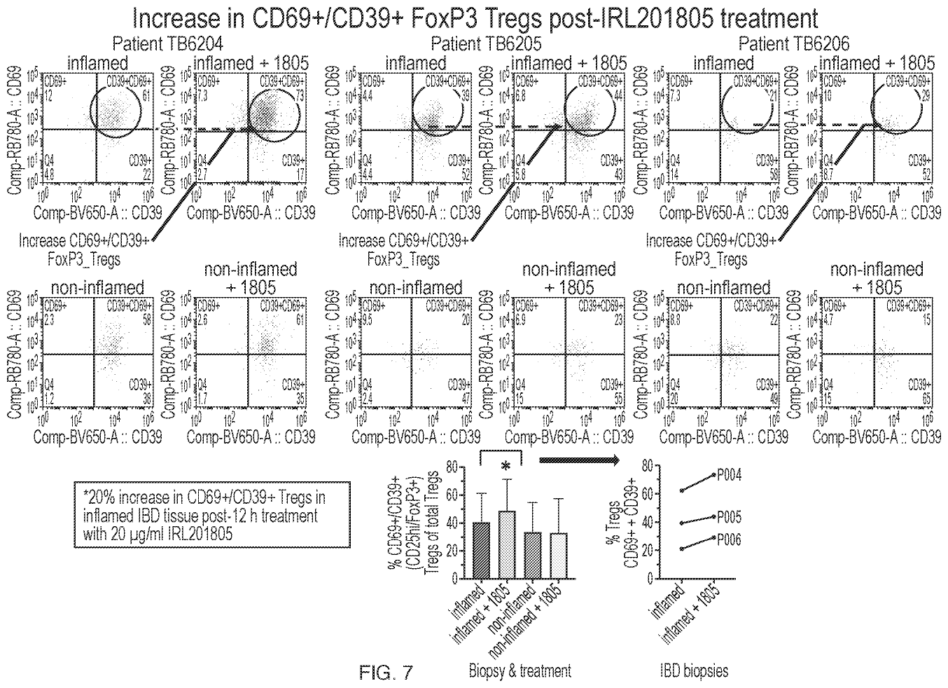

As shown in FIG. 7, exposure of IRL201805 to primary immune cells within inflamed colonic tissue induced a 20% increase in double positive CD69+/CD39+ Tregs compared to the same treatment on non-inflamed tissue from the same patient. In this study, IRL201805 was also shown to have generated increased p-STAT5 in subsets of Immune cells by flow cytometry (data not shown). In previous unpublished data, BiP was also demonstrated to inhibit STAT3 phosphorylation in human lymphocytes. These data suggests that BiP-related peptides would only enhance CD69 activity and the resulting cascade in inflamed tissues, but not in non-inflamed tissues, of IBD patients.

### Example 4 Evaluation of the Effects of Binding Immunoglobulin Protein (BiP) Analogs on T-Cell Activation in PBMCS

### CD69 and CD154 (CD40L) Surface Markers

T-cell activation can be monitored by detecting increased expression of several surface markers, e.g., commonly CD69 and CD154 (CD40L). Expression of CD69 is normally transiently on most T cells, especially T-effector cells, and decreases within 24 h.

In the present study, PBMCs were isolated from two healthy donors. PBMCs were then incubated with IRL201805 (20 μg/mL) for 20-72 h at 37° C. Flow cytometry was used to measure mean fluorescent intensity and assess surface expression of CD69 and CD39 on Tregs from the PBMCs.

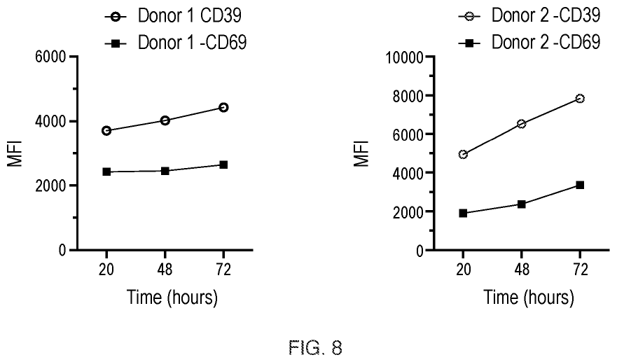

As shown in FIG. 8, IRL201805 treatment induced sustained expression of CD69 expression on CD154-cells and was maintained for at least 72 h. These results are indicative of T cell receptor (TCR)-activated Treg cells.

### JAK-STAT Signaling Pathway

The Janus kinase (JAK)/signal transducer and activator of transcription protein (STAT) (JAK/STAT) pathway orchestrates and controls many of the cytokine and differentiation pathways that control and regulate the immune response. The signaling via the JAK-STAT signaling pathway involves the rapid transmission of signal from the cell membrane to the nucleus followed by a highly organized response and subsequent controlled downregulation and attenuation of the initial signal. Suppressor of cytokine signaling (SOCS) proteins are the primary drivers of signal attenuation. They are induced by cytokine exposure (via STAT) and then act as negative-feedback inhibitors to switch off the signaling cascade. SOCS3 is a potent regulator of cytokine signaling in monocyte/macrophages and T cells.

In the present study, the effects of IRL201805 on JAK-STAT signaling pathway were assessed at the transcript and protein level. PBMCs were isolated from a healthy subject and treated for 24 h with 20 μg/mL IRL201805.

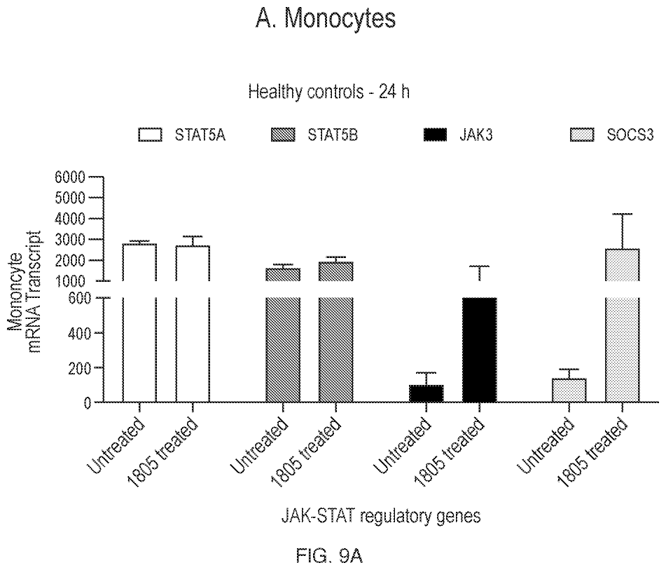

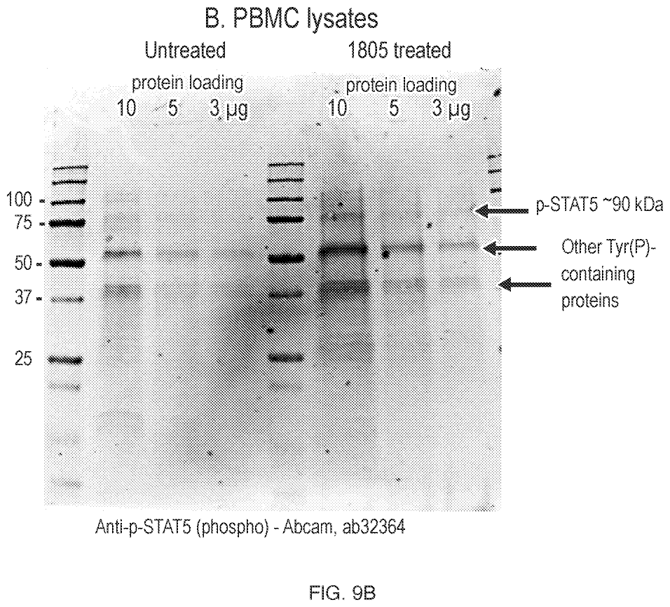

RNAseq analysis was used to assess gene transcript levels of key signaling genes involved in CD69 activation. As shown in FIG. 9A, IRL201805 treatment significantly increased gene transcript levels of JAK3 and SOCS3 by approximately 10-fold measured 24 hour post treatment. IRL201805 did not affect transcript levels of STAT5A or STAT5B in monocytes from three healthy individuals.

Immunoblotting of PBMC cell lysates with recombinant anti-STAT5 (phospho Y694) antibody [E208] (ab32364) (Abcam, ab32364) was used to assess the p-STAT5 levels in from untreated and IRL1805 treated PBMCs. As shown in FIG. 9B, PBMCs isolated from a healthy subject and treated for 24 hours with 20 μg/mL IRL201805 showed increased levels pf p-STAT5 compared to untreated PBMCs.

### FoxP3 Transcription and STAT5 Phosphorylation

In Tregs, CD69 is thought to promote CD39 surface expression, FoxP3 transcription, and STAT5 phosphorylation leading to STAT3 inhibition. The present study assessed the effects of IRL1805 on p-STAT5. IRL201805 generated increased p-STAT5 in subsets of Immune cells. Treatment of PBMCs with IRL201805 led to changes in p-STAT5 status of CD4/CD8 T cells and increased 3-fold the level of FoxP3 transcription (see Table 4).

TABLE 4

| | | CD25^{hi} /CD127^{lo} | % FoxP3 + | |
|---|---|---|---|---|
| Treatment | % CD4^{+ve} | Tregs | Tregs | % CD8^{+ve} |
| Untreated | 10 | 1.8 | 0.3 | 0.3 |
| PHA-treated | 4.4 | 4.3 | 0.6 | 4.2 |
| 1805 treated | 7.1 | 3.2 | 0.9 | 3.2 |

### CD69 Relevant Ligands

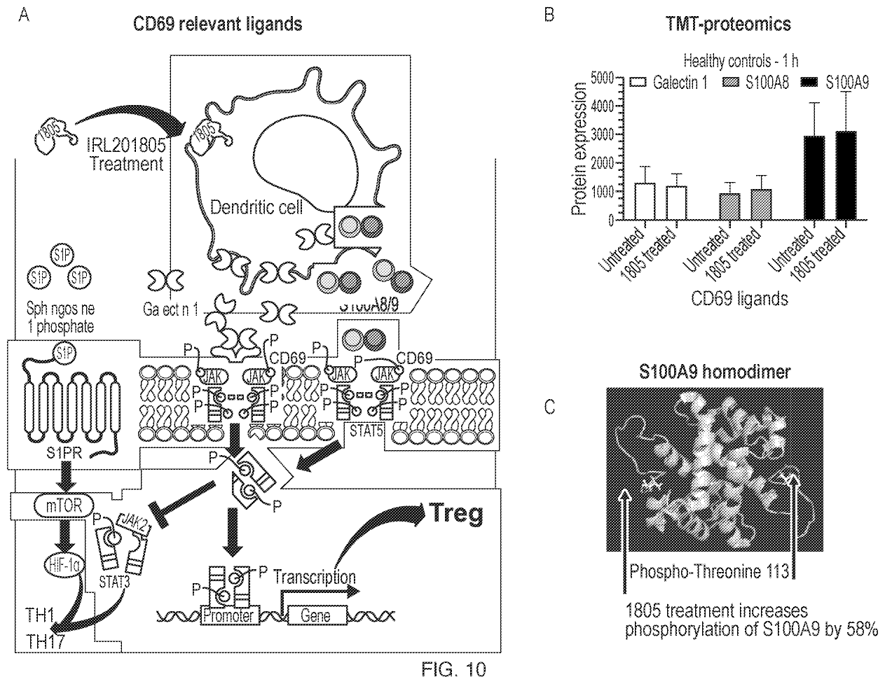

FIG. 10A provides a schematic representation of CD69 relevant ligands. CD69 surface expression is increased upon APC derived MHC class II-TCR activation. TCR engagement induces modest activation of the metabolic sensor mTOR, which intensifies with CD28 and IL-2 signaling, which can promote T cell commitment to TH1, TH2 and TH17 effector cells generation. Membrane SiP1/SiPR1 interaction can sustain the mTOR activation and inhibit differentiation of FoxP3 cells.⁴⁰ DC derived galectin-1 (Gal-1)⁴¹ and S100A8/A942 can bind to CD69 activating JAK3/STAT5 phosphorylation. Phosphorylated STAT5 can translocate to the nucleus and promote differentiation of FoxP3 Tregs and inhibit STAT3 activation and prevent Th17 cell differentiation.

The potential ligands, Gal-1 and S100A8/A9, are known to bind to CD69.43 In order to examine the expression of and influence the ability of T cells to sustain a Treg phenotype, the present study investigated the effects of 1805 on the expression of Gal-1 and S100A8/A9 in APCs (monocytes). Human monocytes isolated from healthy individuals (n=4) were exposed to 1805 for 1 hour at levels of both Gal-1 and S100A8/A9 proteins.

The abundance of Gal-1 and S100A8/A9 protein at the cell surface were detected by tandem mass tag (TMT) quantitative proteomics. As shown in FIG. 10B, Gal-1 and S100A8/A9 expression were not altered by 1805 on the cell surface.

However, the 1805 treatment increased phosphorylation of S100A9 by 58% (FIG. 10C). Mass spec analysis identified increased trimethylation post-translational modifications on threonine 113 on S100A9 post-1805 treatment, a modification which is known to promotes active secretion of S100A9 from myeloid cells.

Surface CD154 and CD69 Activation Markers on T-Lymphocytes from PBMCs

CD69 is commonly detected by flow cytometry as the marker for activated cells most often on lymphocytes and its appearance on the cell surface serves as a biomarker of MHC-TCR interaction between APCs and T-cells. Both CD4 and CD8 T cells express CD69 in homeostatic conditions indicating a number of these cells are constantly exposed to antigen challenge.

The present study assessed the effects of 1805 on the expression of surface CD154 and CD69 activation markers on T-lymphocytes from PBMC of Tregs, Teffs, and CD8 T cells. PBMCs isolated from four healthy donors were cultured with or without 20 μg/mL IRL201805 for 20 hours at 37° C.

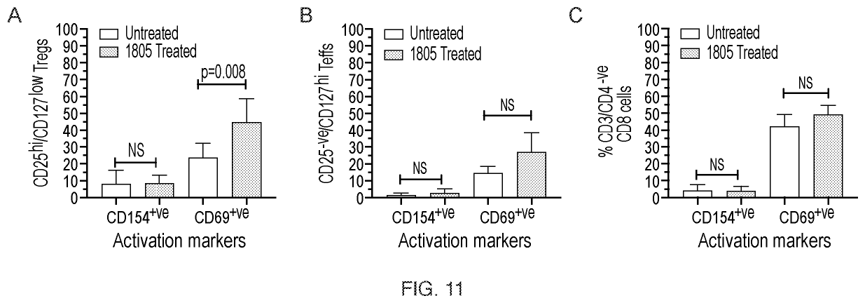

As shown in FIG. 11, the basal level of cell surface CD69 expression was greatest on CD8 T cells (42%, FIG. 11C), followed by Tregs (24%, FIG. 11A) and Teffs (15%, FIG. 11B). These results demonstrate that various subsets of T cells display CD69 on their cell surface. However, 1805 only produced a statistically significant 2-fold increase (p=0.008) on the cell surface of Tregs. These results suggest that Tregs exposed to 1805 are selectively activated.

### IRL201805—Possible Mode of Action

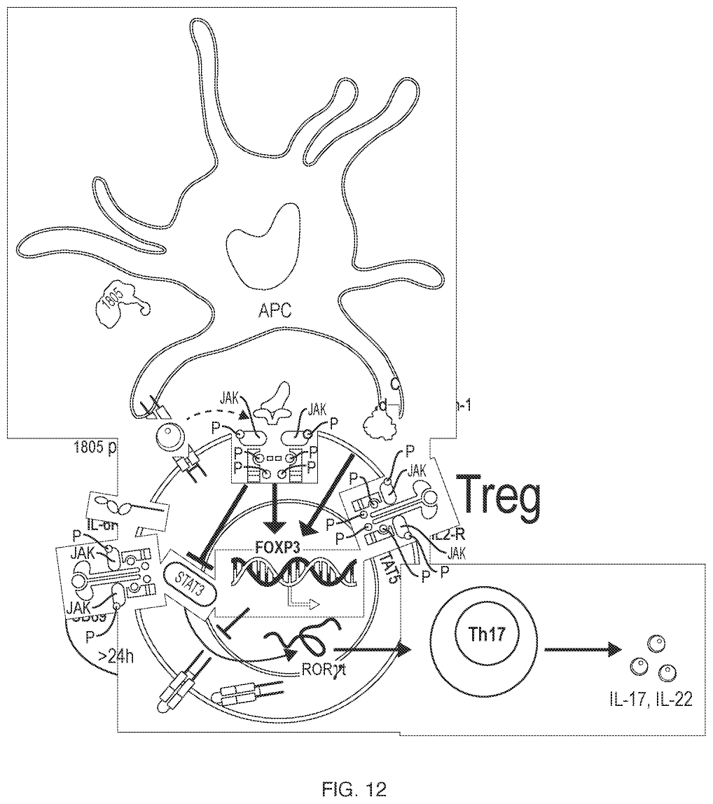

Without being bound to a specific mode of action, FIG. 12 provides a schematic representation of a possible mode of action for the effects of BiP-related peptides such as IRL201805 on CD69 relevant ligands. CD69 receptors are expressed on the membrane of activated regulatory T cells following presentation of 1805 peptide by APCs to the TCR within 2-20 hours. The CD69 receptor binds to ligands on the surface of APCs (e.g., S100A8/A9 or Gal-1). The cytoplasmic tail of CD69 associates with Jak3 and Stat 5 proteins, triggering phosphorylation of Stat5 and its translocation to the nucleus, where it can activate the transcription factor FoxP3, stimulating the differentiation of regulatory T cells.⁴⁴ CD69 engagement can also induce expression of IL-2, TGF-β, and CD39 on the cell surface. These cytokines and receptor may act in an autocrine manner to induce the differentiation of regulatory T cells and tolerogenic APCs. CD69 can inhibit the Th17 differentiation pathway through at least two mechanisms: CD69-activated Stat5 directly inhibits the translocation of Stat3 to the nucleus and, indirectly via FoxP3 activation, antagonizes Stat3-mediated RORγt activation.

### CONCLUSIONS

CD69 has recently been identified as a crucial regulator of intestinal inflammation and a new target molecule for IBD treatment.⁴⁵ Recent in vivo studies with transgenic mice showed that CD69 can limit the immune response and proposed a regulatory function of CD69. CD69 has been shown to have a role in leukocyte migration, in the function of regulatory T cells and resident tissue memory T cells. It has thus been suggested that the stable induction of CD69 expression should lead to the reduced lymphocyte migration to intestinal LP and to the generation of CD69⁺ Treg cells.

The present disclosure provides evidence that BiP-related polypeptides produce stable induction of CD69 expression only in inflamed colon tissues and induces the activation of several CD69 activation markers and CD69 relevant ligands, thus providing a method for modulating an inflammatory response associated with intestinal inflammation and a method of preventing or treating an inflammatory bowel disease (IBD).

### REFERENCES

¹ GBD 2017 Inflammatory Bowel Disease Collaborators (2020). “The global, regional, and national burden of inflammatory bowel disease in 195 countries and territories, 1990-2017: a systematic analysis for the Global Burden of Disease Study 2017.” Lancet Gastroenterol Hepatol. 5:17-30.
² Terlizzi, E. P., et al. (2021). “Health care utilization among U.S. adults with inflammatory bowel disease, 2015-2016.” Natl Health Stat Report 152:1-7.
³ Previously described as SEQ ID NO:3 in PCT application, WO 2019/239126.
⁴ Previously described as SEQ ID NO:4 in PCT application, WO 2019/239126.
⁵ Previously described as SEQ ID NO:1 in PCT application, WO2000/21995.
⁶ Previously described as SEQ ID NO:2 in PCT application, WO2000/21995.
⁷ Previously described as SEQ ID NO:5 in PCT application, WO 2019/239126.

### 8 Isselbacher, et al. (1996). HARRISON'S PRINCIPLES OF INTERNAL MEDICINE, 13 ed., 1814-1882.

9 THE MERCK MANUAL OF DIAGNOSIS AND THERAPY, (2011). 19^{th} Edition, published by Merck Sharp & Dohme Corp., (ISBN 978-0-911910-19-3).
¹⁰ THE ENCYCLOPEDIA OF MOLECULAR CELL BIOLOGY AND MOLECULAR MEDICINE, Robert S. Porter et al. (eds.), published by Blackwell Science Ltd., 1999-2012 (ISBN 9783527600908).
¹¹ MOLECULAR BIOLOGY AND BIOTECHNOLOGY: A COMPREHENSIVE DESK REFERENCE, (1995). Robert A. Meyers (ed.), published by VCH Publishers, Inc. (ISBN 1-56081-569-8).
¹² IMMUNOLOGY, (2006). Werner Luttmann, published by Elsevier.
¹³ JANEWAY'S IMMUNOBIOLOGY, (2014). Kenneth Murphy, Allan Mowat, Casey Weaver (eds.), Taylor & Francis Limited, (ISBN 0815345305, 9780815345305).
¹⁴ LEWIN'S GENES XI, (2014). published by Jones & Bartlett Publishers (ISBN-1449659055).
¹⁵ Michael Richard Green and Joseph Sambrook, (2012). MOLECULAR CLONING: A LABORATORY MANUAL, 4th ed., Cold Spring Harbor Laboratory Press, Cold Spring Harbor, N.Y., USA (ISBN 1936113414).
¹⁶ Davis et al., (2012). BASIC METHODS IN MOLECULAR BIOLOGY, Elsevier Science Publishing, Inc., New York, USA (ISBN 044460149X).
¹⁷ LABORATORY METHODS IN ENZYMOLOGY: DNA, (2013). Jon Lorsch (ed.) Elsevier (ISBN 0124199542).
¹⁸ CURRENT PROTOCOLS IN MOLECULAR BIOLOGY (CPMB), (2014). Frederick M. Ausubel (ed.), John Wiley and Sons (ISBN 047150338X, 9780471503385).
¹⁹ CURRENT PROTOCOLS IN PROTEIN SCIENCE (CPPS), (2005). John E. Coligan (ed.), John Wiley and Sons, Inc.
²⁰ CURRENT PROTOCOLS IN IMMUNOLOGY (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
²¹ Llera, A. S., et al. (2001). “Crystal structure of the C-type lectin-like domain from the human hematopoietic cell receptor CD69” J. Biol. Chem. 276 (10): 7312-7319.
²² Ziegler, S. F., et al. (1994). “The mouse CD69 gene: structure, expression, and mapping to the NK gene complex.” J. Immunology 152 (3): 1228-1236.
²³ Hamann, J., et al. (1993). “Expression cloning of the early activation antigen CD69, a type II integral membrane protein with a C-type lectin domain.” J. Immunology 150 (11): 4920-4927.
²⁴ Radulovic, K. and Niess, J. H. (2015). “CD69 Is the Crucial Regulator of Intestinal Inflammation: A New Target Molecule for IBD Treatment?” J. of Immunology Res. Vol. 2015, Article ID 497056.
²⁵ Testi, R. et al. (1994). “The CD69 receptor: a multipurpose cell-surface trigger for hematopoietic cells.” Immunology Today, 15 (10): 479-483.
²⁶ Alari-Pahissa, E., et al. (2012). “CD69 does not affect the extent of T cell priming.” PLoS ONE, 7 (10): Article ID e48593.
²⁷ Gonza'lez-Amaro, R., et al. (2013). “Is CD69 an effective brake to control inflammatory diseases?” Trends in Molecular Medicine, 19 (10): 625-632.
²⁸ Li, L., et al. (2011). “Identification of M. tuberculosis-specific th1 cells expressing CD69 generated in vivo in pleural fluid cells from patients with tuberculous pleurisy.” PLoS ONE, 6 (8): Article ID e23700.
²⁹ Mori, N., et al. (2011). “Induction of CD69 expression by cagPAI-positive Helicobacter pylori infection.” World Journal of Gastroenterology, 17 (32): 3691-3699.
³⁰ Sancho, D., et al. (2000). “Functional analysis of ligand-binding and signal transduction domains of CD69 and CD23 C-type lectin leukocyte receptors.” J. Immunology, 165 (7): 3868-3875.
³¹ Testi, R., et al. (1989). “T cell activation via Leu-23 (CD69).” J. Immunology, 143 (4): 1123-1128.
³² Cebrian, M., et al. (1988). “Triggering of T cell proliferation through aim, an activation inducer molecule expressed on activated human lymphocytes.” J. Exp. Medicine, 168 (5): 1621-1637.
³³ Santis, A. G., et al. (1992). “Tumornecrosis factor-α production induced in T lymphocytes through the AIM/CD69 activation pathway.” Eur. J. Immunol., 22 (5): 1253-1259.
³⁴ Mcinnes, I. B., et al. (1997). “Interleukin-15 mediates T cell-dependent regulation of tumor necrosis factor-α production in rheumatoid arthritis.” Nature Medicine, 3 (2): 189-195.
³⁵ Radulovic, K. and Niess, J. H. (2015). “CD69 Is the Crucial Regulator of Intestinal Inflammation: A New Target Molecule for IBD Treatment?” J. of Immunology Res. Vol. 2015, Article ID 497056.
³⁶ Id.
³⁷ Powrie, F., et al. (1993). “Phenotypically distinct subsets of CD4+ T cells induce or protect from chronic intestinal inflammation in C. B-17 scid mice.” Int Immunol., 5 (11): 1461-1471.
³⁸ Ostanin D. V., et al. (2009). “T cell transfer model of chronic colitis: concepts, considerations, and tricks of the trade.” Am. J. Physiol. Gastrointest. Liver Physiol., 296 (2): 135-146.
³⁹ Okayasu I., et al. (1990). “A novel method in the induction of reliable experimental acute and chronic ulcerative colitis in mice.” Gastroenterology, 98:694-702.
⁴⁰ Liu, G., et al. (2010). “The S1P(1)-mTOR axis directs the reciprocal differentiation of T(H)1 and T(reg) cells.” Nat. Immunol. 11 (11): 1047-1056.
⁴¹ de la Fuente, H., et al. (2014). “The leukocyte activation receptor CD69 controls T cell differentiation through its interaction with galectin-1.” Mol. Cell Biol. 34:2479-2487.
⁴² Lin, C.-R., et al. (2015). “Glycosylation-dependent interaction between CD69 and S100A8/S100A9 complex is required for regulatory T-cell differentiation.” FASEB J., 29 (12): 5006-5017.
⁴³ Gorabi, A. M., et al. (2020). “The pivotal role of CD69 in autoimmunity.” J. Autoimmunity, 111:102453.
⁴⁴ Cibrian, et al. (2017). European Journal of Immunology, 47:923-1086.
⁴⁵ Radulovic, K. and Niess, J. H. (2015). “CD69 Is the Crucial Regulator of Intestinal Inflammation: A New Target Molecule for IBD Treatment?” J. of Immunology Res. Vol. 2015, Article ID 497056.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain, using no more than routine experimentation, many equivalents to the specific embodiments described herein. Such equivalents are intended to be encompassed by the following claims.

1. A method of preventing or treating an inflammatory bowel disease (IBD) comprising administering to a subject at risk of, or afflicted with, the IBD a therapeutically effective amount of a binding immunoglobulin protein (BiP) or a functional analog thereof.

2. The method of claim 1, wherein the BiP or functional analog thereof has an amino acid sequence selected from the group consisting of: SEQ ID NO:1, SEQ ID NO:2, SEQ ID NO:3, SEQ ID NO:4, and SEQ ID NO:5, or a conservative amino acid substituent thereof.

3. The method of claim 2, wherein the BiP or functional analog thereof has an amino acid sequence of: SEQ ID NO:1.

4. The method of any one of claims 1 to 3, wherein the IBD is selected from the group consisting of: ulcerative colitis, Crohn's disease, and a noninfectious inflammation of the bowel.

5. A therapeutically effective amount of a binding immunoglobulin protein (BiP) or a functional analog thereof for use in preventing or treating an inflammatory bowel disease (IBD).

6. A therapeutically effective amount of a binding immunoglobulin protein (BiP) or a functional analog thereof for use of claim 5, wherein the BiP or functional analog thereof has an amino acid sequence selected from the group consisting of: SEQ ID NO:1, SEQ ID NO:2, SEQ ID NO: 3, SEQ ID NO:4, and SEQ ID NO:5, or a conservative amino acid substituent thereof.

7. A therapeutically effective amount of a binding immunoglobulin protein (BiP) or a functional analog thereof for use of claim 6, wherein the BiP or functional analog thereof has an amino acid sequence of: SEQ ID NO:1.

8. A therapeutically effective amount of a binding immunoglobulin protein (BiP) or a functional analog thereof for use of any one of claims 5 to 7, wherein the IBD is selected from the group consisting of: ulcerative colitis, Crohn's disease, and a noninfectious inflammation of the bowel.


SEQUENCE LISTING (6 sequences)

SEQ ID NO: 1 - protein, 638 aa, synthetic construct
RAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 2 - protein, 648 aa, synthetic construct
MKHHHHHHHMRAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 3 - protein, 639 aa, synthetic construct
MEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAELHHHHHH

SEQ ID NO: 4 - protein, 633 aa, synthetic construct
MEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEL

SEQ ID NO: 5 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 6 - protein, 4 aa, unidentified
KDEL